(19) United States
(12) Patent Application Publication
(10) Pub. No.: US 2011/0189243 A1
(43) Pub. Date: Aug. 4, 2011

(54) PHARMACEUTICAL FORMULATION FOR LOWERING PULMONARY BLOOD PRESSURE

(75) Inventors: Katrin Rimkus, Iserlohn (DE); Frank Muskulus, Laupheim (DE); Sandra Brueck, Ottenhofen (DE); Jana Paetz, Bonn (DE); Dunja Kotschenreuther, Basel (CH)

(73) Assignee: Ratiopharm GMBH

(21) Appl. No.: 13/055,431

(22) PCT Filed: Aug. 7, 2009
(86) PCT No.: PCT/EP09/05749

§ 371 Date: Apr. 22, 2011

(30) Foreign Application Priority Data
Aug. 11, 2008 (DE) 10 2008 037 324.9

(51) Int. Cl. A61K 31/505; C07D 239/34; A61P 9/12; A61K 9/00; B32B 5/16

(52) U.S. Cl. 424/400; 544/318; 514/274; 428/402

(57) ABSTRACT

The invention relates to pharmaceutical formulations for reducing pulmonary blood pressure containing micronised ambrisentan, preferably in the form of an intermediate together with a hydrophilising agent. The invention also relates to methods of preparing pharmaceutical formulations containing micronised ambrisentan.

The invention relates to pharmaceutical formulations for reducing pulmonary blood pressure containing micronised ambrisentan, preferably in the form of an intermediate together with a hydrophilising agent. The invention also relates to methods of preparing pharmaceutical formulations containing micronised ambrisentan.

Ambrisentan is an endothelin receptor antagonist and is approved for the treatment of pulmonary hypertension (high blood pressure in the lungs). As an antagonist, ambrisentan selectively displaces endothelin-1, the most powerful endogenous vasoconstrictor known, from its ET1A receptors and thus cancels out the effect of endothelin-1, so that the vessels dilate, in this way countering the increase in (pulmonary) blood pressure caused by the endothelin, leading in the process to a reduction in (pulmonary) blood pressure.

The IUPAC name for ambrisentan [INN] is (2S)-2-(4,6-dimethylpyrimidin-2-yl)oxy-3-methoxy-3,3-di(phenyl)propanoic acid. The chemical structure of ambrisentan is shown in the (1) below:

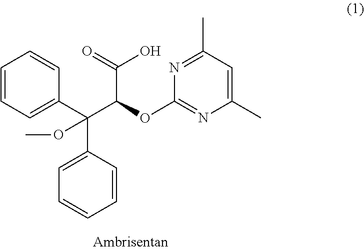

The synthesis of ambrisentan was described by Riechers et al, J. Med. Chem. 39 (11), 2123 (1996) and in WO 96/11914 and leads to a white, crystalline solid.

Ambrisentan is marketed under the trade name Volibris® as film-coated tablets. Volibris contains ambrisentan in crystalline form, with the tablets produced by means of direct compression (see EMEA “Assessment Report for Volibris”, 2008, Procedure No. EMEA/H/C/000839). It has, however, become apparent that tablets produced by means of the direct compression of “untreated” crystalline ambrisentan can be improved with regard to their bioavailability. In addition, it is problematic to obtain a high content of active agent (e.g. 70%) in the tablet with this method. Moreover, it has become apparent that with a low content of active agent (e.g. 15%), the evenness of distribution of the active agent (content unity) ought to be improved.

The objective of the present invention was therefore to overcome the above-mentioned disadvantages. The intention is to provide the active agent in a form which possesses good flowability and makes good compression possible. The resulting tablets should exhibit a high level of hardness and low friability.

The intention is also to provide the active agent in a formulation which possesses good solubility with good storage stability at the same time. In addition, it is intended to achieve a storage stability of 12 months at 40° C. and 75% atmospheric humidity. The impurities after storage under these conditions are intended to be less than 2% by weight, especially less than 1% by weight.

A further aim is that it should be possible to vary the content of active agent over a wide range. Preferably, it is intended to be possible to achieve a content of active agent of 10 to 70% by weight. In addition, the resulting tablet should have a particularly even distribution of active agent; in particular, the intention is for the resulting tablet to have an even distribution of active agent with a low content of active agent (approx. 10 to 20% by weight).

It has unexpectedly been found that the problems can be solved by micronising ambrisentan, preferably by micronising and hydrophilising ambrisentan, especially by micronising, hydrophilising and wet-granulating ambrisentan.

The subject matter of the invention is therefore micronised ambrisentan.

In addition, the subject matter of the invention is an intermediate containing micronised ambrisentan and a hydrophilising agent.

The subject matter of the invention also relates to methods of preparing micronised ambrisentan, or hydrophilised micronised ambrisentan, in the form of the intermediate of the invention.

Finally, the subject matter of the invention also comprises pharmaceutical formulations containing the micronised ambrisentan of the invention, or the micronised and hydrophilised ambrisentan of the invention, in the form of the intermediate.

In the context of this invention, the term “ambrisentan” comprises (2S)-2-(4,6-dimethylpyrimidin-2-yl)oxy-3-methoxy-3,3-di(phenyl)propanoic acid in accordance with formula (1) above. In addition, the term “ambrisentan” comprises all the pharmaceutically acceptable salts and solvates thereof. For all the embodiments of this invention, the term “ambrisentan” preferably means ambrisentan in crystalline form, i.e. preferably more than 90% by weight of the ambrisentan used is present in crystalline form, especially 100%.

The expression “micronised ambrisentan” is used in the context of this invention to designate particulate ambrisentan, which generally has an average particle diameter of 0.1 to 200 μm, preferably 0.5 to 100 μm, more preferably 1 to 50 μm, particularly preferably 1.5 to 30 μm and especially 2 to 20 μm or 1.5 μm to 25 μm and especially 2 μm to 10 μm.

The expression “average particle diameter” relates in the context of this invention to the D₅₀ value of the volume-average particle diameter determined by means of laser diffractometry. In particular, a Malvern Instruments Mastersizer 2000 was used to determine the diameter (wet measurement, 2,000 rpm, ultrasound 60 sec., preferably shading 4 to 13%, preferably dispersion in liquid paraffin, the evaluation using the Fraunhofer method). The average particle diameter, which is also referred to as the D₅₀ value of the integral volume distribution, is defined in the context of this invention as the particle diameter at which 50% by volume of the particles have a smaller diameter than the diameter which corresponds to the D₅₀ value. Similarly, 50% by volume of the particles than have a larger diameter than the D₅₀ value. Analogously, the D₁₀ value of the integral volume distribution is defined as the particle diameter at which 10% by volume of the particles have a smaller diameter than the diameter which corresponds to the D₁₀ value. Accordingly, the D₉₀ value of the integral volume distribution is defined as the particle diameter at which 90% by volume of the particles have a smaller diameter than the diameter which corresponds to the D₉₀ value.

In a preferred embodiment, the ambrisentan of the invention is present in micronised and hydrophilised form, namely in the form of an intermediate containing micronised ambrisentan and a hydrophilising agent. In particular, the intermediate of the invention consists substantially of micronised ambrisentan and hydrophilising agent. The expression “substantially” in this case indicates that small amounts of solvent etc. may also be present where applicable.

The “hydrophilising agent” in the context of this invention is generally a substance which is capable of accumulating on ambrisentan (chemically or physically) and increasing the hydrophilicity of the surface.

The hydrophilising agent may be hydrophilic polymers. This means polymers which possess hydrophilic groups. Examples of suitable hydrophilic groups are hydroxy, amino, carboxy, sulphonate. In addition, the hydrophilic polymer which can be used in order to prepare the intermediate preferably has a number-average molecular weight of 1,000 to 500,000 g/mol, more preferably 2,000 to 50,000 g/mol. When the polymer used as the hydrophilising agent is dissolved in water in an amount of 2% by weight, the resulting solution preferably has a viscosity of 1 to 20 mPas, more preferably 1 to 5 mPas, even more preferably 2 to 4 mPas, measured at 25° C. and determined in accordance with Ph. Eur., 6th edition, chapter 2.2.10.

Furthermore, the hydrophilising agent also encompasses solid, non-polymeric compounds, which preferably contain polar side groups. Examples of these are sugar alcohols or disaccharides.

The intermediate of the invention may, for example, comprise the following hydrophilic polymers as hydrophilising agents: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC), microcrystalline cellulose; polyvinyl pyrrolidone, polyvinyl alcohol, polymers of acrylic acid and their salts, polyacrylamide, polymethacrylates, vinyl pyrrolidone-vinyl acetate copolymers (such as Kollidon® VA64, BASF), polyoxyethylene/polyoxypropylene block polymer (Poloxamer®), gelatine, polyalkylene glycols, such as polypropylene glycol or preferably polyethylene glycol, and mixtures thereof.

Similarly, sugar alcohols and/or disaccharides such as mannitol, sorbitol, xylitol, isomalt, sucrose, lactose, glucose, fructose, maltose and mixtures thereof can preferably be used as hydrophilising agents. The term “sugar alcohols” in this context also includes monosaccharides.

In the context of this invention, it has further been unexpectedly found that in particular “brittle” hydrophilising agents can be used especially advantageously.

Hydrophilising agents can generally be classified with regard to the change in the shape of the particles under compression pressure (compaction): plastic hydrophilising agents are characterised by plastic deformation, whereas when compressive force is exerted on brittle excipients, the particles tend to break into smaller particles. Brittle behaviour on the part of the hydrophilising agent can be quantified by the increase in the surface area in a compressed part. In the art, it is customary to classify the brittleness in terms of the “yield pressure”. According to a simple classification, the values for the “yield pressure” here are low for plastic substances but high in the case of friable substances, on the other hand [Duberg, M., Nyström, C., 1982. Studies on direct compression of tablets VI. Evaluation of methods for the estimation of particle fragmentation during compaction. Acta Pharm. Suec. 19, 421-436; Humbert-Droz P., Mordier D., Doelker E. <<Méthode rapide de détermination du comportement à la compression pour des études de préformulation>>, Pharm. Acta Helv., 57, 136-143 (1982)). The “yield pressure” describes the pressure that has to be reached for the substance (i.e. the hydrophilising agent) to begin to flow plastically.

The “yield pressure” is preferably calculated using the reciprocal of the gradient of the Heckel plot, as described in York, P., Drug Dev. Ind. Pharm. 18, 677 (1992). The measurement in this case is preferably made according to the “ejected tablet” method at 25° C. and a deformation rate of 0.1 mm/s.

In the context of the present invention, a hydrophilising agent is deemed a brittle hydrophilising agent if it has a “yield pressure” of at least 80 MPa, preferably 90 to 300 MPa.

Examples of preferred brittle hydrophilising agents are microcrystalline cellulose, lactose and sucrose.

In a preferred embodiment, the intermediate of the invention contains micronised ambrisentan and hydrophilising agent, the weight ratio of micronised ambrisentan to hydrophilising agent being 50:1 to 1:5, more preferably 20:1 to 1:1, even more preferably 15:1 to 2:1, especially 15:1 to 5:1.

It is preferable that the type and amount of the hydrophilising agent are selected such that at least 50% of the surface of the resulting intermediate particles is covered with hydrophilising agent, more preferably at least 60% of the surface, particularly preferably at least 80% of the surface, especially at least 95% of the surface.

The intermediate of the invention may optionally contain an emulsifier and/or pseudo-emulsifier instead of or preferably in addition to the hydrophilising agent. For this purpose, the pseudo-emulsifiers explained in more detail below are preferably used.

The subject matter of the invention is consequently a method of preparing the micronised ambrisentan of the invention or the intermediate of the invention. In this embodiment, the intermediate of the invention contains micronised ambrisentan and hydrophilising agent and/or pseudo-emulsifier, the weight ratio of micronised ambrisentan to hydrophilising agent and/or pseudo-emulsifier being 50:1 to 1:5, more preferably 20:1 to 1:1, even more preferably 15:1 to 2:1, especially 15:1 to 5:1.

Micronised ambrisentan in accordance with the invention is usually obtainable by milling.

In a preferred embodiment, the invention relates to a milling process for preparing the intermediate of the invention, comprising the steps of

(a1) mixing crystalline ambrisentan and hydrophilising agent, and
(b1) milling the mixture from step (a1).

Crystalline (non-micronised) ambrisentan and hydrophilising agent are mixed in step (a1). The mixture is milled in step (b1). The mixing may take place before or even during the milling, i.e. steps (a1) and (b1) may be performed simultaneously.

The milling conditions are selected such that at least 50% of the surface of the resulting intermediate particles is covered with hydrophilising agent, more preferably at least 60% of the surface, particularly preferably at least 80% of the surface, especially at least 95% of the surface.

The milling is generally performed in conventional milling apparatuses, such as in a ball mill, air jet mill, pin mill, classifier mill, cross beater mill, disk mill, mortar grinder, rotor mill. An air jet mill is preferably used.

The milling time is usually 0.5 minutes to 1 hour, preferably 2 minutes to 50 hours, more preferably 5 hours to 30 hours.

The process conditions in this embodiment are preferably selected such that the resulting intermediate particles have a volume-average particle diameter (D₅₀) of 0.1 to 250 μm, more preferably 0.5 to 50 μm, especially 1 to 25 μm or 1 μm to 20 μm.

The process described above leads to the intermediate of the invention containing micronised ambrisentan and hydrophilising agent. The subject matter of the invention therefore also relates to intermediates obtainable by this method.

The inventors of the present application have found that the problems underlying the invention can also be solved in an alternative embodiment by an intermediate containing micronised ambrisentan, optionally in combination with ambrisentan in the form of a solid solution, and hydrophilising agent.

In an alternative embodiment, the invention consequently relates to a process for preparing the intermediate containing ambrisentan in micronised form (and optionally partially in the form of a solid solution) and a hydrophilising agent. The preparation preferably takes the form of “pellet-layering”. The subject matter of the invention is thus a process comprising the steps of

(a2) suspending the crystalline ambrisentan and the hydrophilising agent in a solvent or mixture of solvents, and
(b2) spraying the solution from step (a2) onto a substrate core.

In step (a2), ambrisentan, preferably ambrisentan and the hydrophilising agent described above are suspended in a solvent or mixture of solvents, i.e. ambrisentan remains at least partially in crystalline form.

Suitable solvents are. for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and DMSO is used.

Suitable hydrophilising agents in this alternative embodiment are in particular modified celluloses, such as HPMC, sugar alcohols, such as mannitol and sorbitol, and polyethylene glycol, especially polyethylene glycol with a molecular weight of 2,000 to 10,000 g/mol.

In step (b2), the suspension from step (a2) is sprayed onto a substrate core. Suitable substrate cores are particles consisting of pharmaceutically acceptable excipients, especially “neutral pellets”. The pellets preferably used are those which are obtainable under the trade name Cellets® and which contain microcrystalline cellulose.

Step (b2) is preferably performed in a fluidised bed dryer, such as a Glatt GPCG 3 (Glatt GmbH, Germany).

The process conditions in this second embodiment are preferably selected such that the resulting intermediate particles have a volume-average particle diameter (D₅₀) of 50 to 750 μm, more preferably 100 to 500 μm.

The micronised ambrisentan of the invention and the intermediate of the invention (i.e. the hydrophilised and micronised ambrisentan of the invention or the hydrophilised ambrisentan in crystalline form) are usually employed to prepare a pharmaceutical formulation.

The subject matter of the invention is therefore a pharmaceutical formulation containing micronised ambrisentan of the invention or intermediate of the invention and pharmaceutical excipients.

These are the excipients with which the person skilled in the art is familiar, such as those which are described in the European Pharmacopoeia.

Examples of excipients used are disintegrants, anti-stick agents, emulsifiers, pseudo-emulsifiers, fillers, additives to improve the powder flowability, glidants, wetting agents, gelling agents and/or lubricants.

The ratio of active agent to excipients is preferably selected such that the resulting formulations contain 1 to 70% by weight, more preferably 2 to 30% by weight, especially 5 to 20% by weight micronised ambrisentan and 30 to 99% by weight, more preferably 70 to 98% by weight, especially 80 to 95% by weight pharmaceutically acceptable excipients.

In these ratios specified, the amount of hydrophilising agent optionally used to prepare the intermediate of the invention is counted as an excipient. This means that the amount of active agent refers to the amount of micronised ambrisentan contained in the intermediate.

It has become apparent that a purposive selection of disintegrant is particularly preferable in solving the problems described above.

In a preferred embodiment, the pharmaceutical formulation of the invention contains

(i) 1 to 70% by weight, more preferably 2 to 30% by weight, especially 5 to 20% by weight micronised ambrisentan and
(ii) 0.5 to 25% by weight, more preferably 2 to 20% by weight, especially 3 to 15% by weight or 1 to 25% by weight, more preferably 3 to 20% by weight, especially 5 to 15% by weight disintegrants, based on the total weight of the formulation.

In addition, the pharmaceutical formulation preferably contains one or more of the above-mentioned excipients.

“Disintegrants” is the term generally used for substances which accelerate the disintegration of a dosage form, especially a tablet, after it is placed in water. Suitable disintegrants are, for example, organic disintegrants such as carrageenan, croscarmellose, sodium carboxymethyl starch and crospovidone. Alkaline disintegrants are preferably used. The term “alkaline disintegrants” means disintegrants which, when dissolved in water, produce a pH level of more than 7.0.

More preferably, inorganic alkaline disintegrants are used, especially salts of alkali metals and alkaline earth metals. Preferred examples here are sodium, potassium, magnesium and calcium. As anions, carbonate, hydrogen carbonate, phosphate, hydrogen phosphate and dihydrogen phosphate are preferable. Examples are sodium hydrogen carbonate, sodium hydrogen phosphate, calcium hydrogen carbonate and the like.

Crospovidone and/or croscarmellose are particularly preferably used as disintegrants, especially in the above-mentioned amounts.

In a further preferred embodiment, the pharmaceutical formulation additionally contains

(iii) anti-stick agents, preferably in an amount of 0.1 to 5% by weight, more preferably 0.5 to 3% by weight, based on the total weight the formulation.

The anti-stick agent (iii) is especially important when the micronised ambrisentan is used as the intermediate of the invention.

“Anti-stick agents” is usually understood to mean substances which reduce agglomeration in the core bed. Examples are talcum, silica gel, polyethylene glycol (preferably with 2,000 to 10,000 g/mol weight-average molecular weight) and/or glycerol monostearate. Examples of preferred anti-stick agents are talcum and polyethylene glycol (Mg 3,000-6,000 g/mol), carrageenan.

In a further preferred embodiment, the pharmaceutical formulation additionally contains a

(iv) pseudo-emulsifier, preferably in an amount of 0.1 to 5% by weight, more preferably 0.5 to 3% by weight, based on the total weight the formulation.

Pseudo-emulsifiers are usually (preferably polymeric) substances which, when added to a solution, increase the viscosity of that solution. Preferably, the addition of 5% by weight of pseudo-emulsifier to distilled water at 20° C. leads to an increase in the viscosity of at least 1%, preferably at least 2%, in particular at least 5%.

The pseudo-emulsifiers used are preferably plant gums. Plant gums are polysaccharides of natural origin which cause the above-mentioned viscosity increase.

Examples of suitable pseudo-emulsifiers are agar, alginic acid, alginate, chicle, dammar, mallow extracts, gellan (E 418), guar gum (E 412), gum arabic (E 414), gum from psyllium seed husks, gum from spruce resin, locust bean gum (E 410), karaya (E 416), glucomannan (E 425), obtained from the konjac root, tara gum (E 417), gum traganth (E 413), xanthan gum (E 415), preferably prepared by bacterial fermentation, and/or lecithin.

Gum arabic, agar and/or lecithin are preferably used.

Possible emulsifiers are anionic emulsifiers, e.g. □soaps, preferably alkali salts of higher fatty acids □salts of bile acid (alkali salts); cation-active emulsifiers, e.g. □benzalconium chloride, □cetyl pyridinium chloride, □cetrimide; non-ionic emulsifiers, e.g. □sorbitan derivatives, especially sorbitan monolaurate, polyoxythylene-(20)-sorbitan-monolaurate, □polyethylene glycol derivatives/polyoxyethylene derivative, especially polyoxyethylene-(20)-sorbitan monostearate, polyoxythylene stearate or polyoxyethylene stearyl ether. In addition, partial fatty acid esters of polyhydric alcohols can be used, such as glycerol monostearate, □fatty acid ester of sucrose, □fatty acid ester of polyglycol or □casein. Furthermore, mixtures of the above-mentioned substances can be used.

In addition to components (i) to (iv), the formulation of the invention may also contain further, above-mentioned pharmaceutical excipients. These will be explained in more detail below.

The formulation of the invention preferably contains fillers. “Fillers” generally means substances which serve to form the body of the tablet in the case of tablets with small amounts of active agent (e.g. less than 70% by weight). This means that fillers “dilute” the active agents in order to produce an adequate tableting mixture. The normal purpose of fillers, therefore, is to obtain a suitable tablet size.

Examples of preferred fillers are lactose, lactose derivatives, starch, starch derivatives, treated starch, talcum, chitin, cellulose and derivatives thereof, calcium phosphate, sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride. Prosolv® (Rettenmaier & Söhne, Germany) can likewise be used.

Fillers are normally used in an amount of 1 to 80% by weight, more preferably 30 to 60% by weight, based on the total weight of the formulation.

One example of an additive to improve the powder flowability is disperse silicon dioxide, e.g. known under the trade name Aerosil®. Preferably, silicon dioxide is used with a specific surface area of 50 to 400 m²/g, determined by gas adsorption in accordance with Ph. Eur., 6th edition 2.9.26.

Additives to improve the powder flowability are generally used in an amount of 0.1 to 3% by weight, based on the total weight of the formulation.

In addition, lubricants may be used. Lubricants are generally used in order to reduce sliding friction. In particular the intention is to reduce the sliding friction found during tablet pressing between the punch moving up and down in the die and the die wall, on the one hand, and between the edge of the tablet and the die wall, on the other hand. Suitable lubricants are, for example, stearic acid, adipic acid, sodium stearyl fumarate and/or magnesium stearate.

Lubricants are generally used in an amount of 0.1 to 3% by weight, based on the total weight of the formulation.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient. For example, PEG 4000—if used as a hydrophilising agent—is not additionally used as an anti-stick agent (even though PEG 4000 also exhibits a release effect). Similarly, microcrystalline cellulose—if used as a hydrophilising agent—is not additionally used as a disintegrant, for example (even though microcrystalline cellulose also exhibits a certain disintegrating effect).

The pharmaceutical formulation of the invention is preferably pressed into tablets. In the state of the art, direct pressing of an ambrisentan formulation is proposed (cf. EMEA “Assessment Report for Volibris”, 2008, Procedure No. EMEA/H/C/000839).

It has, however, become apparent that the properties of the resulting tablets can be improved if the pharmaceutical formulation of the invention is subjected to wet granulation or suspension-granulation before being pressed into a tablet.

The subject matter of the invention is thus a process comprising the steps of
(I) providing micronised ambrisentan or preferably intermediate of the invention and pharmaceutical excipients,
(II) wetting or suspending the substances from step (I) with or in a granulation solution,
(III) granulating the wetted substances or granulating them with suspended substances and
IV) drying and, where appropriate, screening the granules obtained,

In step (I), ambrisentan of the invention or intermediate of the invention and pharmaceutical excipients are prepared. The pharmaceutical excipients are preferably the excipients described above.

The substances are preferably mixed. The mixing can be performed in conventional mixers. In order to ensure an even distribution, mixing in intensive mixers is preferable. The mixing may, for example, be performed in compulsory mixers or free-fall mixers. Alternatively, the mixing can occur during steps (II) and (III).

The substances from step (I) are wetted with a granulation liquid or suspended in a granulation liquid. Suitable granulation liquids are, for example, water, alcohols and mixtures thereof. A mixture of water and ethanol is preferred.

Steps (I) to (IV) can be carried out in standard granulation apparatuses. The “one-pot process” or the “fluidised-bed process” are preferred here.

In the one-pot process, the substances from step (I) are wetted and granulated with granulation liquid. Steps (II) and (III) are preferably performed concurrently. The granules are then dried and optionally screened. A suitable granulating machine is, for example, Diosna P1/6.

In the fluidised-bed process, the substances from step (I) are suspended in granulation liquid and sprayed to dry them. The mixing, wetting, granulating and drying are performed in one operation. The granules are then optionally screened. A suitable fluidised-bed granulator is, for example, a Glatt GPCG 3.

In a preferred embodiment, the granulation conditions are selected such that the resulting particles (granules) have a volume-average particle size (d(₅₀) value) of 50 to 600 μm, more preferably 100 to 500 μm, even more preferably 150 to 400 μm, especially 200 to 350 μm.

In addition, the granulation conditions are preferably selected such that the resulting granules have a bulk density of 0.2 to 0.85 g/ml, more preferably 0.3 to 0.8 g/ml, especially 0.4 to 0.7 g/ml. The Hausner factor is usually in the range from 1.03 to 1.3, more preferably from 1.04 to 1.20 and especially from 1.04 to 1.15. The “Hausner factor” in this context means the ratio of compacted density to bulk density.

The granules resulting from step (IV) can be further processed into pharmaceutical dosage forms. For this purpose, the granules are filled into sachets or capsules, for example. The granules resulting from step (IV) are preferably pressed into tablets. The pressing step (V) will be described below. The subject matter of the invention thus relates to tablets obtainable by compressing a granulated material obtained from step (IV).

In step (V) of the method, the granules obtained in step (IV) are pressed into tablets, i.e. the step involves compression into tablets. The compression can be performed with tableting machines known in the state of the art.

In step (V) of the method, pharmaceutical excipients may optionally be added to the granules from step (IV).

The amounts of excipients added in step (V) usually depend on the type of tablet to be produced and the amount of excipients which were already added in step (I). During compression, the additives to improve powder flowability described above and the lubricants described above are preferably used.

The tablets produced by the method of the invention may be tablets which can be swallowed unchewed (non-film-coated or preferably film-coated). They may likewise be chewable tablets or dispersible tablets. “Dispersible tablet” here means a tablet to be used for producing an aqueous suspension for swallowing.

In the case of tablets which are swallowed unchewed, it is preferable that they be coated with a film layer. For this purpose, the methods of film-coating tablets which are standard in the state of the art may be employed. The above-mentioned ratios of active agent to excipient, however, relate to the uncoated tablet.

The tableting conditions are preferably selected such that the resulting tablets have a ratio of tablet height to weight of 0.005 to 0.3 mm/mg, particularly preferably 0.05 to 0.2 mm/mg.

In addition, the resulting tablets preferably have a hardness of 35 or 50 to 200 N, particularly preferably 40 to 100 N or 80 to 150 N. The hardness is determined in accordance with Ph. Eur. 6.0, section 2.9.8.

In addition, the resulting tablets preferably have a friability of less than 10%, particularly preferably less than 8%. The friability is determined in accordance with Ph. Eur. 6.0, section 2.9.7.

Finally, the tablets of the invention usually have a “content uniformity” of 85 to 115% of the average content, preferably 90 to 110%, especially 95 to 105% of the average content. The “content uniformity” is determined in accordance with Ph. Eur. 6.0, section 2.9.6.

The release profile of the tablets of the invention according to the USP method after 10 minutes usually indicates a content released of at least 30%, preferably at least 50%, especially at least 70%.

The above details regarding hardness, friability, content uniformity and release profile preferably relate here to the non-film-coated tablet.

Apart from the preferred wet granulation described above, it is also possible as an alternative for the pharmaceutical formulation of the invention to be subjected to dry granulation before being pressed into a tablet.

The subject matter of the invention is thus alternatively a method comprising the steps of

(I-T) preparing the micronised ambrisentan of the invention or the intermediate of the invention and one or more pharmaceutical excipients (especially those described above);
(II-T) compacting into flakes; and
(III-T) granulating the flakes.

In step (I-T), ambrisentan and excipients are preferably mixed. The mixing can be performed in conventional mixers. Alternatively, it is possible that the micronised and preferably hydrophilised ambrisentan is initially mixed with only part of the excipients (e.g. 50 to 95%) before compacting (II), and that the remaining part of the excipients is added after the granulation step (III-T). In the case of multiple compacting, the excipients should preferably be mixed in before the first compacting step, between multiple compacting steps or after the last granulation step.

In step (II-T) of the alternative method of the invention, the mixture from step (I-T) is compacted into flakes. It is preferable here that it should be dry compacting, i.e. the compacting is preferably performed in the absence of solvents, especially in the absence of organic solvents.

The compacting conditions in step (II-T) are preferably selected such that the flakes have a density of 0.75-1.1 g/cm³.

The term “density” here preferably relates to the “pure density” (i.e. not to the bulk density or compacted density). The pure density can be determined with a gas pycnometer. The gas pycnometer is preferably a helium pycnometer; in particular, the AccuPyc 1340 helium pycnometer from the manufacturer Micromeritics, Germany, is used.

The compacting is preferably carried out in a roll granulator.

The rolling force is preferably 2 to 50 kN/cm, more preferably 4 to 30 kN/cm, especially 10 to 25 kN/cm.

The gap width of the roll granulator is, for example, 0.8 to 5 mm, preferably 1 to 4 mm, more preferably 1.5 to 3 mm, especially 1.8 to 2.8 mm.

The compacting apparatus used preferably has a cooling means. In particular, the cooling is such that the temperature of the compacted material does not exceed 50° C., especially 40° C.

In step (III-T) of the method the flakes are granulated. The granulation can be performed with methods known in the state of the art.

In a preferred embodiment, the granulation conditions are selected such that the resulting particles (granules) have a volume-average particle size (d(₅₀) value) of 50 to 600 μm, more preferably 100 to 500 μm, even more preferably 150 to 400 μm, especially 200 to 350 μm.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

In a preferred embodiment, the method is adapted such that multiple compacting occurs, with the granules resulting from step (III-T) being returned one or more times to the compacting process (II-T). The granules from step (III-T) are preferably returned 1 to 5 times, especially 2 to 3 times.

The granules resulting from step (III-T) can be further processed into pharmaceutical dosage forms, as described above in connection with wet granulation. For this purpose, the granules are filled into sachets or capsules, for example. The granules resulting from step (III-T) are preferably pressed into tablets.

In the case of tablets which are swallowed unchewed, it is preferable that they be coated with a film layer. For this purpose, the methods of film-coating tablets which are standard in the state of the art may be employed. The above-mentioned ratios of active agent to excipient, however, relate to the uncoated tablet.

For film-coating, macromolecular substances are preferably used, such as modified celluloses, polymethacrylates, polyvinyl pyrrolidone, polyvinyl acetate phthalate, zein and/or shellack.

HPMC is preferably used, especially HPMC with a number-average molecular weight of 10,000 to 150,000 g/mol and/or an average degree of substitution of —OCH₃ groups of 1.2 to 2.0.

The thickness of the coating is preferably 10 to 100 μm.

The invention will now be explained with reference to the following examples.

### EXAMPLES

### Example 1

### Micronisation and Direct Pressing

20 g crystalline ambrisentan was micronised for 30 minutes at 350 rpm in a PM 100 ball mill (ex Retsch) together with 2 g polaxomer as a hydrophilising agent.

The micronised active agent was suspended in water together with 2 g Povidon/4 g gum arabic. This suspension was used for the granulation of 100 g Avicel®, 50 g lactose, 20 g carboxymethyl starch (Diosna P 1).

The granules were dried and then screened (Comil U5; 1.25 mm).

The granules were added with 1 g Aerosil®, 2 g magnesium stearate and 30 g Avicel® to a mixture suitable for tableting and mixed for a further 5 minutes in a free-fall mixer.

The tablets had a hardness of 40-100 N, combined with a friability of less than 10%.

### Example 2

### Micronisation and Wet Granulation

20 g crystalline ambrisentan was micronised in a Jetmill Alpine 50 AS air jet mill at 4-6 bar (ex Hosokawa). The active agent was then mixed for 5 min. with 2 g PEG (Lutrol®) as a hydrophilising agent in order to ensure an even coating over the particles.

The micronised active agent was suspended in water together with 0.5 g HPMC/1 g gum arabic. This suspension was used for the granulation of 90 g corn starch, 12 g crospovidone (Diosna P 1).

The granules were dried and then screened (Comil U5; 1.25 mm).

Both 0.8 g Aerosil® and 1.6 g magnesium stearate, and also 20 g starch 1500 were added to the granules. Everything was mixed in a free-fall mixer for a further 5 minutes to form a mixture suitable for tableting.

The tablets had a hardness of 40-100 N, combined with a friability of less than 10%.

The tablets were coated with 4 g HPMC (Pharmacoat® 603), 0.5 g titanium dioxide, 0.5 g talcum and 0.3 PEG in a drum coater (Lödige LHC 25) from an aqueous solution.

### Example 3

### Micronisation

2 g crystalline ambrisentan was milled for 30 minutes at 350 rpm in a Retsch ball mill.

The following average particle sizes were found:

d₉₀=63 μm; d₅₀=18 μm, d₁₀=5 μm

### Example 4

### Wet Granulation

0.77 g ambrisentan according to Example 3
15.00 g corn starch

### 1.05 g PVP

7.50 g water

### 1.68 g Aerosil®

2.77 g corn starch
0.21 g magnesium stearate
0.15 g sodium stearyl fumarate

The ambrisentan, corn starch and PVP were granulated with water. The granules were dried for 60 minutes at 40° C. Aerosil®, corn starch and magnesium stearate were screened through a 1,000 μm screen, added to the granules and mixed for 3 minutes. Pruv® was added and mixed again. The mixture was pressed into tablets of 155 mg each. The content of active agent was 5 mg.

### Example 5

### Direct Tableting

0.20 g ambrisentan according to example 3
5.34 g corn starch
0.06 g magnesium stearate
0.20 g Prosolv® (siliconised MCC)

### 0.07 g Aerosil®

Ambrisentan and corn starch were mixed together for 15 minutes in the Turbula T10B mixer at 32 rpm. Magnesium stearate was added and mixed for a further 3 minutes. Then Prosolv® and Aerosil® were added in order to improve the flowability. The mixture was pressed directly into tablets of 147 mg each.

### Example 6

### Direct Tableting

0.77 g ambrisentan according to example 3
19.60 g calcium hydrogen carbonate
0.42 g sodium carboxymethyl starch
0.21 g magnesium stearate

Ambrisentan, calcium hydrogen carbonate and sodium carboxymethyl starch were weighed in together and mixed for 15 minutes. Magnesium stearate was added and mixed for a further 3 minutes. The mixture was pressed directly into tablets of 140 mg each. The content of active agent was 5 mg.

### Example 7

### Direct Tableting

0.77 g ambrisentan according to example 3
19.39 g Microcelac® (75% lactose monohydrate and 25% microcrystalline cellulose)
0.63 g croscarmellose sodium
0.21 g magnesium stearate

Ambrisentan, Microcelac® and croscarmellose sodium were weighed in together and mixed for 15 minutes. Magnesium stearate was added and mixed for a further 3 minutes. The mixture was pressed directly into tablets of 140 mg each. The content of active agent was 5 mg.

### Example 8

### Micronisation and Direct Tableting

0.77 g ambrisentan
19.39 g Microcelac® (75% lactose monohydrate and 25% microcrystalline cellulose)
0.63 g croscarmellose sodium
0.21 g magnesium stearate

Ambrisentan and Microcelac® were milled for 30 min in a ball mill at 350 rpm. Croscarmellose sodium and magnesium stearate were added to the mixture and mixed for a further 3 minutes. The mixture was pressed directly into tablets of 140 mg each. The content of active agent was 5 mg.

### Example 9

### Micronisation and Direct Tableting

0.77 g ambrisentan

### 4.81 g MCC

14.16 g calcium hydrogen carbonate
1.05 g sodium carboxymethyl starch
0.21 g magnesium stearate

Ambrisentan and MCC were milled for 30 min in a ball mill at 350 rpm. Sodium carboxymethyl starch and calcium hydrogen phosphate were added to the mixture and mixed for a further 10 minutes. After that, magnesium stearate was added and mixed for 3 minutes. The mixture was pressed directly into tablets of 140 mg each. The content of active agent was 5 mg.

### Example 10

### Micronisation and Direct Tableting

0.77 g ambrisentan
4.81 g sucrose
14.16 g calcium hydrogen carbonate
1.05 g sodium carboxymethyl starch
0.21 g magnesium stearate

Ambrisentan and sucrose were milled for 30 min in a ball mill at 350 rpm. Sodium carboxymethyl starch and calcium hydrogen phosphate were added to the mixture and mixed for a further 10 minutes. After that, magnesium stearate was added and mixed for 3 minutes. The mixture was pressed directly into tablets of 140 mg each. The content of active agent was 5 mg.

### Example 11

### Micronisation and Wet Granulation

0.77 g ambrisentan
4.81 g MCC (part 1)
9.14 g MCC (part 2)

### 1.05 g PVP

7.50 g water

### 1.20 g Aerosil®

2.77 g calcium hydrogen carbonate
1.05 g sodium carboxymethyl starch
0.21 g magnesium stearate

Ambrisentan and MCC (part 1) were milled for 30 min at 350 rpm in a ball mill. The milled material, MCC (part 2) and PVP were used to prepare granules with water. The granules were dried overnight at 40° C. Aerosil®, calcium hydrogen phosphate, sodium carboxymethyl starch and magnesium stearate were screened through a 1,000 μm screen, added to the granules and mixed for 3 minutes. The mixture was pressed into tablets of 140 mg each. The content of active agent was 5 mg.

### Example 12

### Micronisation and Wet Granulation

0.77 g ambrisentan
4.81 g MCC (part 1)
9.14 g MCC (part 2)

### 1.05 g PVP

14.50 g water

### 1.20 g Aerosil®

2.77 g calcium hydrogen carbonate
1.05 g sodium carboxymethyl starch
0.21 g magnesium stearate

Ambrisentan and MCC (part 1) were milled for 30 min at 350 rpm in a ball mill. The milled material was used to prepare a suspension with water. This was sprayed onto MCC (part 2) and PVP, and granules were prepared. The granules were dried overnight at 40° C. Aerosil®, calcium hydrogen phosphate, sodium carboxymethyl starch and magnesium stearate were screened through a 1,000 μm screen, added to the granules and mixed for 3 minutes. The mixture was pressed into tablets of 140 mg each. The content of active agent was 5 mg.

1. Micronised ambrisentan.

2. The ambrisentan as claimed in claim 1, wherein the average particle diameter is 0.1 to 50 μm.

3. An intermediate containing ambrisentan as claimed in claim 1 and a hydrophilising agent.

4. The intermediate as claimed in claim 3 wherein the weight ratio of ambrisentan to hydrophilising agent is 1:1 to 25:1, preferably 5:1 to 15:1.

5. The intermediate as claimed in claim 3, obtainable by jointly micronising ambrisentan and a hydrophilising agent.

6. The intermediate as claimed in claim 3, wherein the hydrophilising agent is a hydrophilic polymer or a sugar alcohol.

7. The intermediate as claimed in claim 3, wherein the hydrophilising agent is a brittle compound with a yield pressure of at least 80 MPa.

8. An intermediate containing micronised ambrisentan, optionally in combination with a solid solution of ambrisentan, obtainable by a process comprising the steps of
(a) suspending ambrisentan and hydrophilising agent in a solvent or mixture of solvents, and
(b) spraying the suspension from step (a) onto a core.

9. A pharmaceutical formulation containing micronised ambrisentan as claimed in claim 1, and pharmaceutical excipients.

10. The pharmaceutical formulation as claimed in claim 9, containing a pseudo-emulsifier.

11. The pharmaceutical formulation as claimed in claim 9, containing an alkaline disintegrant.

12. A method of preparing a pharmaceutical formulation comprising the steps of
(I) providing the micronised ambrisentan as claimed in claim 1, and pharmaceutical excipients,
(II) wetting or suspending the substances from step (I) with or in a granulation solution,
(III) granulating the wetted or suspended substances, and
(IV) drying and, where appropriate, screening the granules obtained.

13. A tablet, obtainable by compressing granules as claimed in claim 12.

14. The tablet as claimed in claim 13 with a hardness of 40 150 N and a friability of less than 10%.

15. The tablet as claimed in claim 13 with a “content uniformity” of 85 to 115% of the average content.
